(19) United States
(12) Patent Application Publication
(10) Pub. No.: US 2012/0128770 A1
(43) Pub. Date: May 24, 2012

(54) TREATMENT OF INSULIN RESISTANCE AND OBESITY BY STIMULATING GLP-1 RELEASE

(75) Inventors: Harald S. Hansen, Vanlose (DK); Thi Ai Diep, Copenhagen Nv (DK); Jens Juul Holst, Hellerup (DK); Mette Marie Rosenkilde, Soborg (DK); Niels Wellner Andersen, Kobenhavn N (DK)

(73) Assignee: Kobenhavns Universitet, Copenhagen K (DK)

(21) Appl. No.: 13/380,086

(22) PCT Filed: Jun. 24, 2010
(86) PCT No.: PCT/DK2010/050161

§ 371 Date: Jan. 31, 2012

Related U.S. Application Data
(60) Provisional application No. 61/220,097, filed on Jun. 24, 2009.

(30) Foreign Application Priority Data
Jun. 24, 2009 (DK) PA 2009 70037

(51) Int. Cl. A61K 31/231; A61K 9/48; C12N 5/02; A61K 9/52; C07C 69/58; A61P 3/10; A61K 31/22; A61K 9/64

(52) U.S. Cl. 424/456; 514/549; 514/546; 514/547; 424/451; 424/463; 424/457; 560/224; 435/375

(57) ABSTRACT

The present invention relates to 1 or 2 C16-C18 acyl glycerol based compounds which are capable of activating G-protein coupled receptor 119 and thereby stimulate GLP-1 release. Compounds of the present invention are useful in the prophylaxis and/or treatment of metabolic disorders and complications thereof, such as, type 2 diabetes mellitus (T2DM), obesity, insulin resistance, and cardiovascular disease.

### FIELD OF INVENTION

The present invention relates to compounds which are capable of activating G-protein coupled receptor 119 and thereby stimulate GLP-1 release. Compounds of the present invention are useful in the prophylaxis and/or treatment of metabolic disorders and complications thereof, such as, type 2 diabetes mellitus (T2DM), obesity, insulin resistance, and cardiovascular disease.

### BACKGROUND OF INVENTION

T2DM is a multifaceted metabolic disease characterized by a combination of resistance to insulin action and an inadequate compensatory insulin secretory response resulting in increased concentration of glucose in the blood [1]. The prolonged hyperglycemia of T2DM is associated with increased risk of developing severe vascular injury in eyes, kidney, heart and the nervous system. The risk of developing T2DM increases with age, obesity and lack of physical activity [1].

In the USA, the number of people with diagnosed diabetes (2007) now reaching 17.5 million is growing by approximately 1 million per year. More than 90% of those diagnosed diabetic have T2DM. This increase in the prevalence of T2DM is seen all over the world, and it is partly associated with the increased prevalence of obesity. The total estimated cost of diabetes in USA in 2007 is $174 billion, including $116 billion in excess medical expenditures and $58 billion in reduced national productivity [2]. A very large group of non-diabetic people have impaired fasting glucose levels (5.6-6.9 mmol/l) and they are now referred to as having pre-diabetes indicating their relatively high risk for developing T2DM [1].

The medical treatment of T2DM focuses on obtaining a tight control of blood glucose levels in order to minimize the risk of developing macrovascular and microvascular complications. A number of different antidiabetica exists. One relatively new group of antidiabetic medicines—the incretin-based medicines—enhances the glucose-stimulated insulin release by increasing the presence of the incretin hormone glucagon-like peptide-1 (GLP-1). GLP-1 is secreted from the hormone-producing L-cells of the intestinal tract in response to unknown components in the meal and GLP-1 increases the glucose-stimulated insulin release from the pancreas [3]. Furthermore, GLP-1 also reduces gastric emptying, increases satiety and can result in weight loss [4;5]. The incretin effect is strongly reduced in patients with T2DM [6]. The incretin-based medicines increase the plasma concentration of GLP-1 either by inhibiting the GLP-1 catabolic enzyme DPP4 (called incretin enhancers), or by providing stable GLP-1 receptor agonists (called incretin mimetics) [7]. A third way of increasing GLP-1 secretion is to activate the GPR119, an orphan receptor that is found in the small intestine and in the pancreas [8;9]. It is known that stimulation of this receptor will increase GLP-1 secretion from intestinal cells in vitro [10] and in rodents in vivo [8]. Oleoylethanolamide and lysophosphatidylcholine have been reported to be endogenous activators of GPR119 [11;12]. It is known that exogenous oleoylethanolamide can inhibit food intake [13-15]. However, this anorexic effect of exogenous oleoylethanolamide is not mediated via activation of intestinal GPR119 but probably via activation of intestinal PPARalpha [16;17].

### SUMMARY OF INVENTION

It has surprisingly been found that certain compounds can activate GPR119 in the intestine and thereby stimulate the intestinal GLP-1 release. Through stimulation of GLP-1 release the compounds can be used for treatment and/or prophylaxis of metabolic syndrome, diabetes-2, obesity, insulin resistance and cardiovascular disease.

Therefore, in a first aspect the invention relates to a compound selected from the group consisting of

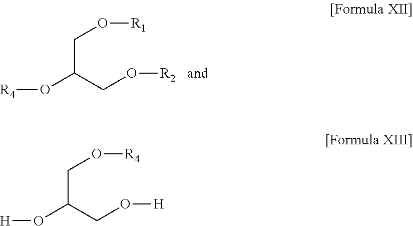

wherein R₁ and R₂ are individually selected from the group consisting of H, and C2-C8 acyl groups,
and wherein R₄ is selected from the group consisting of a C16 or C18 acyl group, for use in the treatment and/or prophylaxis of metabolic syndrome, cardiovascular disease, diabetes-2, obesity or insulin resistance.

The invention also relates to use of a compound according to the invention for activating G-protein coupled receptor 119.

In one aspect the invention relates to a pharmaceutical composition comprising a compound according to the invention and a pharmaceutically acceptable carrier, excipient and/or diluent.

Furthermore, there is provided a capsule comprising a compound according to the invention or a solvate of said compound, wherein the capsule is made of one or several from the group consisting of gelatine, a plant based gelling substance such as carrageenans, starch, cellulose, modified starch, and modified cellulose, such as hydroxypropyl methylcellulose, and derivatives of any of these. These capsules protect the compounds from hydrolysis in the stomach so that they can reach the target in the intestine.

The compounds may be formulated as a combination product comprising; (A) a compound according to the invention or a pharmaceutical composition according to the invention, and (B) another therapeutic agent that is useful in the treatment of metabolic syndrome diabetes type 2, obesity, insulin resistance, and/or cardiovascular disease, for simultaneous, successive or separate administration.

Furthermore, the invention relates to a method of activating G-protein coupled receptor 119 by administering a compound according to the invention to a cell expressing said receptor.

Also provided is a method of stimulating the release of GLP-1 in the gastrointestinal tract, in the pancreas and in the brain of a subject in need thereof comprising administering to said subject a therapeutically effective amount of a compound according the invention.

In an important aspect, the invention relates to a food or animal feed product comprising a compound selected from the group consisting of

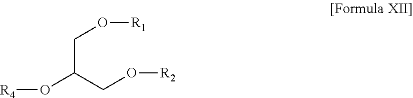

wherein R₁ and R₂ are individually selected from the group consisting of H, and C2-C8 acyl groups,
and wherein R₄ is selected from the group consisting of a C16 or C18 acyl group.

Most of the compounds of the invention are edible and are in the shape of oils that can be readily mixed into food and feed products and can replace a fraction of the oil or fat normally present in the food or feed.

Furthermore, the invention relates to use of a compound according to the invention as a low calorie fat substitute.

An aspect of the invention relates to use of a compound according to the invention for preparation of a dietary supplement, a food or feed product, or a beverage product for helping to sustain energy, helping control appetite, helping control blood sugar levels, reducing the risks associated with metabolic syndrome, reducing the risk associated with obesity and diabetes, reducing the risk associated with diabetes, helping to maintain healthy glucose and fat metabolism, or for helping to normalise production and release of GLP1 necessary for healthy glucose and fat metabolism, in a subject during and/or between meals or feedings comprising said dietary supplement, a food or feed product, or a beverage product.

A related aspect relates to a method for helping to sustain energy, helping control appetite, helping control blood sugar levels, reducing the risks associated with metabolic syndrome, reducing the risk associated with obesity and diabetes, reducing the risk associated with diabetes, helping to maintain healthy glucose and fat metabolism, or for helping to normalise production and release of GLP1 necessary for healthy glucose and fat metabolism, said method comprising administering to a subject a dietary supplement, a food or feed product, or a beverage product comprising a compound of the invention during and/or between meals or feedings.

The compounds of the invention, which are supplemented with the compounds of the invention, may provide a general improvement to human health. First and foremost, the compounds of the invention can protect against the harmful health effects associated with metabolic syndrome. It is also contemplated that the compounds of the invention can protect against the harmful health effects associated with type 2 diabetes, and against the harmful health effects associated with obesity.

In certain embodiments, the compounds of the invention can help reverse the harmful health effects associated with metabolic syndrome. It is also believed that the compounds products of the invention can help reverse the harmful health effects associated with obesity.

As shown by the examples the compounds of the invention can help normalise production and release of incretins involved in glucose homeostasis and fat metabolism. Normalisation of production and release of incretins is important to human health. The compounds of the invention can help stimulate production and release of incretins involved in glucose homeostasis and fat metabolism. More specifically the compounds of the invention can help normalise production and release of GLP1 necessary for efficient glucose homeostasis and fat metabolism. The compounds of the invention can help normalise production and release of GLP1 necessary for human health.

These beneficial health effects can also be brought about by using the food and feed products of the invention.

### DESCRIPTION OF DRAWINGS

FIG. 1. Ligand-induced GPR119 activation in CHO-K1 cells. Five different ligands were tested. Oleoylethanolamide, H-1: 2-oleoylglycerol, H-2: 1-oleoyleglycerol, H-4: oleic acid, and H-5: 2-palmitoylglycerol. cAMP was measured by a HTRF kit from CisBio International. Three experiments (each in duplicate) were performed using oleoylethanolamide as positive control in each experiment. All dose-response curves were normalized to the efficacy of OEA in each experiment, and the data are shown as ligand-induced activation above basal receptor activity.

FIG. 2. Ligand-induced GPR119 activation in COS-7 cells.

Three different ligands were tested: (C) oleoylethanolamide (OEA), (A) 2-oleoylglycerol (2OG), and (D) oleic acid, by measurement of cAMP accumulation. Briefly, COS-7 cells were transiently transfected with the human GPR119 receptor (squares) or empty vector (negative control, triangle). The cells were seeded in plates one day after transfection, incubated for 24 hours with 2 μCi/ml of ³H-adenine. At the day of the cAMP measurement, the cells incubated with the ligands for 25 minutes, lysed, and the cAMP purified using Dowex and alumina columns. The data represent sum-curves of three independent experiments (n=3), performed in duplicates. All dose-response curves were normalized to the efficacy of OEA in each experiment, and the data are shown as ligand-induced activation above basal receptor activity.

FIG. 3. 2-OG-induced activation of GPR119-mediated cAMP formation.

Briefly, COS-7 cells were transiently transfected with human GPR119 receptor (squares) or empty vector (negative control, triangle). The cells were seeded in plates one day after transfection, incubated for 24 hours with 2 μCi/ml of ³H-adenine. At the day of the cAMP measurement, the cells were incubated with the ligand for 25 minutes, lysed, and the cAMP purified using Dowex and alumina columns. The data represent sum-curves of the 7 independent experiments, performed in duplicates. All dose-response curves were normalized to the efficacy of OEA in each experiment, and the data are showed as ligand-induced activation above basal receptor activity.

FIG. 4. Stimulation of tGLP-1 release by 2OG in the intestinal lumen. Eight healthy male volunteers were given enteral feeding by duodenal tube. Each volunteer was given three different liquid meals (two grams of 2OG(▴), 1.54 grams of oleic (∘) acid or vehicle (⋄)) on three different days. The vehicle consisted of 50 ml glycerol plus 5 ml ethanol. Data are mean values±SEM and were analysed by repeated measurements ANOVA. The incremental area under the curve (iAUC) for the first 25 min was significantly increased (p=0.010) with the following values 0.07±0.02 nM×25 min for 2OG, −0 0.04±0.02 nM×25 min for oleic acid, and 0.03±0.02 nM×25 min for vehicle.

### DEFINITIONS

Treatment: “Treatment” can be performed in several different ways, including curative, ameliorating and as prophylaxis. Curative treatment generally aims at curing a clinical condition, such as a disease, which is already present in the treated individual. Ameliorating treatment generally means treating in order to improve in an individual an existing clinical condition. Prophylactic treatment generally aims at preventing a clinical condition from occurring or from developing further.

### DETAILED DESCRIPTION OF THE INVENTION

It has surprisingly been found that compounds selected from the group consisting of

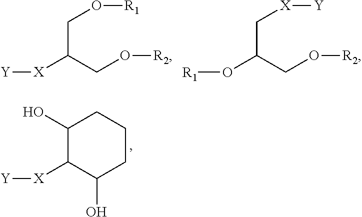

### and Y—OH,

activate GPR119 in the intestine and thereby stimulate the intestinal GLP-1 release. Said compounds can be used for the prophylaxis and/or treatment of diabetes-2, obesity, insulin resistance, and cardiovascular disease.

Accordingly, in a first main aspect the invention relates to a compound selected from the above defined group for use as a medicament.

In another main aspect the invention relates to a method of treating metabolic syndrome, diabetes-2, obesity, insulin resistance, and/or cardiovascular disease comprising administering an effective amount of a compound as defined above to a subject in need of such treatment.

In a further aspect the invention relates to compounds as defined above capable of activating G-protein coupled receptor 119.

The present invention in its broadest aspect is directed to a compound selected from the group consisting of

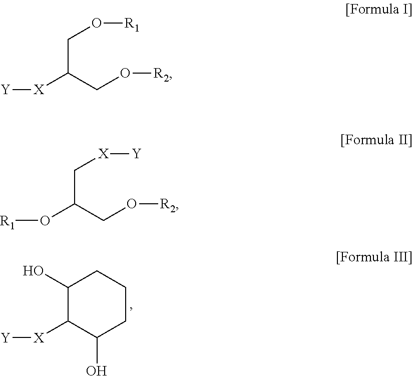

and Y—OH [Formula IV],
wherein R₁ and R₂ are individually selected from the group consisting of
H, and

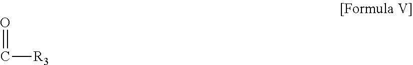

wherein R₃ is selected from the group consisting of alkyl groups comprising 1-12 carbon atoms,
Y—X is selected from the group consisting of

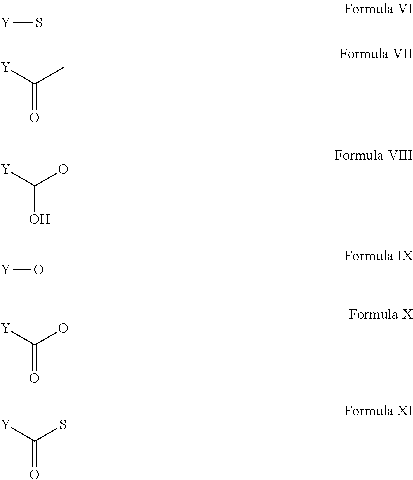

Y is selected from the group consisting of an alkyl group comprising a carbon chain comprising at least 12 carbon atoms and comprising 0, 1, 2, 3 or 4 double bonds,
for use as a medicament.

In a preferred embodiment R₁ and/or R₂ are

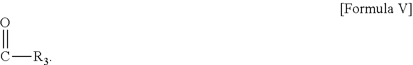

In a more preferred embodiment R₃ is selected from an alkyl group consisting of 1-12 carbon atoms, such as 1-11, for example 1-10, such as 1-9, for example 1-8, such as 1-7, for example 1-6, such as 1-5, for example 1-4, such as 1-3 carbon atoms.

In an even more preferred embodiment R₃ is selected from an alkyl group consisting of 1-2 carbon atoms.

In a preferred embodiment X is O.

In one embodiment Y is selected from a group consisting of an alkyl group comprising a carbon chain consisting of 12, 14, 16, 18, or 20 carbon atoms. Said carbon chain further comprises 0, 1, 2, 3, or 4 double bonds.

In another embodiment Y is selected from a group consisting of an alkyl group comprising a carbon chain consisting of 13, 15, 17, or 19 carbon atoms. Said carbon chain further comprising 0, 1, 2, 3, or 4 double bonds.

In a preferred embodiment Y is selected from a group consisting of an alkyl group comprising a carbon chain consisting of 12 carbon atoms and 0 double bonds, an alkyl group comprising a carbon chain consisting of 14 carbon atoms and 0 double bonds, an alkyl group comprising a carbon chain consisting of 14 carbon atoms and 1 double bond, an alkyl group comprising a carbon chain consisting of 16 carbon atoms and 0 double bonds, an alkyl group comprising a carbon chain consisting of 16 carbon atoms and 1 double bond, an alkyl group comprising a carbon chain consisting of 18 carbon atoms and 0 double bonds, an alkyl group comprising a carbon chain consisting of 18 carbon atoms and 1 double bond, an alkyl group comprising a carbon chain consisting of 18 carbon atoms and 2 double bonds, an alkyl group comprising a carbon chain consisting of 18 carbon atoms and 3 double bonds, or an alkyl group comprising a carbon chain consisting of 20 carbon atoms and 0 double bonds.

In a more preferred embodiment Y is selected from a group consisting an alkyl group comprising a carbon chain consisting of 18 carbon atoms and 0 double bonds, an alkyl group comprising a carbon chain consisting of 18 carbon atoms and 1 double bond, an alkyl group comprising a carbon chain consisting of 18 carbon atoms and 2 double bonds, or an alkyl group comprising a carbon chain consisting of 18 carbon atoms and 3 double bonds, or an alkyl group comprising a carbon chain consisting of 18 carbon atoms and 4 double bonds.

In an even more preferred embodiment Y is an alkyl group comprising a carbon chain consisting of 18 carbon atoms and 1 double bond.

In a preferred embodiment R₁ and/or R₂ are

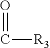

wherein R₃ is an alkyl group consisting of 1-2 carbon atoms, X is O, and Y is an alkyl group comprising a carbon chain consisting of 18 carbon atoms and 1 double bond.

In another preferred embodiment the compound is Y—OH

### Preferred Compounds of the Invention

A preferred aspect of the invention relates to compounds selected from the group consisting of

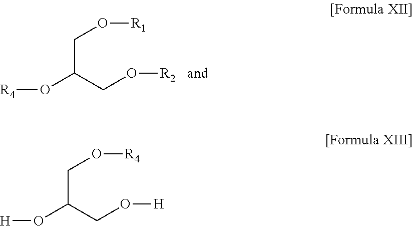

wherein R₁ and R₂ are individually selected from the group consisting of H, and C2-C8 acyl groups,
and wherein R₄ is selected from the group consisting of a C16 or C18 acyl group, for use in the treatment and/or prophylaxis of metabolic syndrome, cardiovascular disease, diabetes-2, obesity or insulin resistance.

Formula XII is a combination of above identified Formula I and Formula X. Likewise Formula XIII is a combination of above identified Formula II and Formula X. These compounds are glycerols with a long chain fatty acid in the 2 (Formula XII) or 1 (Formula XIII) position and optionally with short chain fatty acids in the 1 and 3 positions (Formula XII) and the 2 and 3 positions (Formula XIII) respectively.

Preferably R₁ and R₂ are linear and saturated as most edible short chain fatty acids are.

In one embodiment one of R₁ and R₂ is CH2-CH3 and the other is —H. Preferably, both R₁ and R₂ are CH2-CH3. Such acetylated glycerols will be hydrolysed in the stomach to the non-acetylated forms. R₁ and/or R₂ may also be —H.

Preferably, R₄ is linear. In a preferred embodiment, R₄ is unsaturated. In certain embodiments R₄ is C16 acyl group comprising 0, 1 or 2 double bonds. Examples of such R₄ groups include palmitic and palmitoleic. In other, preferred embodiments, R₄ is C18 acyl group comprising 0, 1, 2, 3, or 4 double bonds. Examples of such R₄ groups include oleic, linoleic, alpha-linoleic, elaidic, gamma linoleic, or stearidonic, preferably oleic. 2-palmitoyl-glycerol has been shown to be an activator of GPR119 by the present inventors.

In a more preferred embodiment the compound is 1-oleoylglycerol. 1-oleoylglycerol is chemically stable in aqueous solution. In aqueous solution 1-OG isomerises spontaneously into 2-OG. Therefore, 1-OG may be regarded as a prodrug of 2-OG. When taken orally, the isomerisation starts already in the mouth and may continue in the stomach and intestine. This isomerisation process starts already in the mouth and continues into the stomach and the intestinal lumen. As 2-monoacylglycerols are taken up very rapidly, the equilibrium (1-OG12-OG) may be shifted even more in favour of 2-OG in the intestine as 2-OG is taken up.

Similarly, it is expected that 1-palmitoly-glycerol can act as a prodrug for 2-palmitoyl-glycerol.

Different 1-monoacylglycerols are widely commercially used as emulsifiers in the food, cosmetic and pharmaceutical industry [20] and 1-oleoylglycerol is cheap and easily available. 1-OG is generally recognised as safe by the FDA.

In yet a preferred embodiment the compound is 1-acetyl-2-oleoylglycerol, 1-acetyl-2-alpha-linoleylglycerol, 1-acetyl-2-elaidoylglycerol, and 1-acetyl-2-gamma-linoleylglycerol. Among these, 1-acetyl-2-oleoylglycerol is most preferred.

Even more preferred is 1,3 diacetyl-2-oleylglycerol, 1,3 diacetyl-2-linoleylglycerol, 1,3 diacetyl-2-alpha-linoleylglycerol, 1,3 diacetyl-2-elaidoylglycerol, and 1,3diacetyl-2-gamma-linoleylglycerol. Among these, 1,3 diacetyl-2-oleylglycerol is most preferred.

Also preferred are glycerols with a C18 unsaturated acid in the 2 position. Examples of these are 2-oleylglycerol, linoleylglycerol, 2-elaidoylglycerol, and 2-gamma-linoleylglycerol.

In a most preferred embodiment the compound is 2-oleoylglycerol. 2-oleylglycerol was found to activate GPR119 nearly as potently as oleoylethanolamide (Example 1). 2-oleylglycerol can relatively easy be synthesized from olive oil or canola oil by the use of 1,3-specific lipases [19].

In a further embodiment, the compound is a triacylglycerol-like type of prodrug used to circumvent the chemical instability of 2-oleylglycerol. Such a prodrug is chemically stable and may be hydrolyzed to 2-oleylglycerol in the intestinal lumen. Preferred prodrugs are those with short-chain acyl (C2-C8) groups in the 1 and 3 positions. Short chain acyl groups in the 1 and 3 positions have the advantage that they provide less calories than triglycerides with two or three long fatty acids.

In a further embodiment the compound is the prodrug 1,3-di-acetyl-2-oleoylglycerol.

The compounds of the present invention are capable of activating GPR119 and stimulating GLP-1 release in the gastrointestinal tract.

By activation/activating GPR 119 is meant binding to GPR119 and causing formation of cAMP and a subsequent increase in the level of intracellular cAMP.

GPR119 is found both in the intestine where it is involved in release of GLP-1 and in the pancreatic islet where it is involved in release of insulin [21]. GPR119 is also found in the brain (Chu et al, (2008) Endocrinology, 149; 2038-2047).

GPR119 is also known as SNORF25, RUP3, GPCR2, 19AJ, OSGPR116, and glucose-dependent insulinotropic receptor (Overton H A et al (2008): Br. J. Pharmacol. 153; 76-81).

The compounds of the invention in one preferred embodiment are defined as having an EC50 of 50 μm or lower, such as 40 μM or lower, for example 30 μm or lower, such as 20 μM or lower, where EC50 is defined as the concentration of said compound needed for half-maximal activation of GPR119 measured by cAMP-formation. The EC50 as defined above is preferably determined in an assay as described in Example 1. The EC50 may be 18 μM or lower, such as 15 μM or lower, for example 12 μM or lower, such as 10 μM and lower, for example 8 μM or lower such as 5 μM or lower.

The compounds of the invention are useful as medicaments for prophylaxis, and/or treatment of metabolic syndrome, diabetes-2, obesity, insulin resistance, and cardiovascular disease. The effect on GLP-1 release may be confirmed in an in vivo experiment such as described in Example 2.

In one embodiment the compounds of the invention are used for the treatment and/or prophylaxis of cardiovascular disease alone.

In a preferred embodiment, the compound according to the invention is for the treatment and/or prophylaxis of metabolic syndrome, diabetes-2, obesity or insulin resistance. Each of these disorders may be treated alone or in combination with one of the others. Likewise, the compounds of the invention may be used in the treatment of metabolic syndrome.

Accordingly the invention relates to a compound according to the invention for the treatment and/or prophylaxis of diabetes-2.

In another aspect it relates to a compound of the invention for the treatment and/or prophylaxis of obesity.

In another aspect it relates to a compound of the invention for the treatment and/or prophylaxis of metabolic syndrome.

Finally, the invention relates to a compound according to the invention for the treatment and/or prophylaxis of insulin resistance.

Accordingly, the invention relates to the use of one or more of the compounds as defined above as a medicament.

The invention also relates to the use of one or more of the compounds for the manufacture of a medicament.

### Pharmaceutical Compositions

Further, the invention relates to a pharmaceutical composition comprising a compound as defined above, or a solvate of said compound, and a pharmaceutically acceptable carrier, excipient and/or diluent. Suitable excipients and/or diluents can be, but are not limited to lecithin, bile salts, Macrogol, sorbitan esters, polysorbates, ethanol, glycerol, medium-length triglycerides.

Strategies in formulation development of medicaments and compositions based on the compounds of the present invention generally correspond to formulation strategies for drug products. Potential problems and the guidance required to overcome these problems are dealt with in several textbooks, e.g. Handbook of Pharmaceutical Excipients; Rowe R C, Sheshey P. J. and Owen S. C.; 5th. edition, Pharmaceutical Press, 2006, Physicochemical Principles of Pharmacy; Florence A. T. and Attwood D.; 3rd edition, Palgrave 1998; Almen Farmaci; Kristensen H. G.; 3. oplag, HCø tryk, 2004

Formulations of the compounds of the invention can be prepared by techniques known to the person skilled in the art. The formulations may contain pharmaceutically acceptable carriers and excipients including microspheres, liposomes, microcapsules, nanoparticles or the like.

For formation of liposomes and nanoliposomes an emulsifier should be added. Examples of emulsifiers include lecithin, such as lecithin from egg yolk or soybean, honey, mustard powder, proteins and low molecular weight emulsifiers. Lipid vesicles are formed when e.g. phospholipids such as lecithin are placed in water and consequently form one bilayer or a series of bilayers, each separated by water molecules, once enough energy is supplied. Liposomes can e.g. be created by shaking or sonicating phospholipids in water.

The medicament of the invention comprises an effective amount of one or more of the compounds as defined above, or a composition comprising a compound as defined above, in combination with pharmaceutically acceptable additives. Such medicament may suitably be formulated for oral and intravenous administration routes. Oral administration is preferred.

Injectables are usually prepared either as liquid solutions or suspensions, solid forms suitable for solution in, or suspension in, liquid prior to injection. The preparation may also be emulsified. The active ingredient is often mixed with excipients which are pharmaceutically acceptable and compatible with the active ingredient. Suitable excipients are, for example, water, saline, dextrose, glycerol, ethanol or the like, and combinations thereof. In addition, if desired, the preparation may contain minor amounts of auxiliary substances such as wetting or emulsifying agents, pH buffering agents, or which enhance the effectiveness or transportation of the preparation.

The compounds according to the invention may preferably be administered by oral formulations.

The compounds according to the invention are administered in a manner compatible with the dosage formulation, and in such amount as will be therapeutically effective. The quantity to be administered depends on the subject to be treated, including, e.g. the weight and age of the subject, the disease to be treated and the stage of disease.

Some of the compounds of the present invention are sufficiently active, but for some of the others, the effect will be enhanced if the preparation further comprises pharmaceutically acceptable additives and/or carriers. Such additives and carriers will be known in the art. In some cases, it will be advantageous to include a compound, which promotes delivery of the active substance to its target.

The preferred oral formulation comprising a compound according to the invention can be presented as units suitable for oral administration, such as capsules, tablets, or cachets.

In a preferred embodiment of the invention the oral formulation comprising a compound according to the invention is presented as capsules.

Suitable capsule materials can be, but are not limited to gelatine, a plant based gelling substance such as carrageenans, starch, cellulose, modified starch, and modified cellulose, such as hydroxypropyl methylcellulose.

Capsules can be formulated for sustained and/or controlled release.

Capsules can have an enteric coating. Different enteric coating polymers for enteric coated capsules can be, but are not limited to; Cellulose acetate phthalate (CAP), Methyl acrylate-methacrylic acid copolymers, Cellulose acetate succinate, Hydroxy propyl methyl cellulose phthalate, Hydroxy propyl methyl cellulose acetate succinate (hypromellose acetate succinate), Polyvinyl acetate phthalate (PVAP), and Methyl methacrylate-methacrylic acid copolymers.

Suitable coatings for duodenum delivery can be, but are not limited to; EUDRAGIT® L 100-55 which contains an anionic copolymer based on methacrylic acid and ethyl Acrylate, EUDRAGIT® L 30 D-55 which is the aqueous dispersion of an anionic copolymer based on methacrylic acid and ethyl acrylate.

Suitable coatings for jejunum/Ileum delivery can be, but are not limited to; Eudragit® L100, Eudragit® S100, Eudragit® NE 30D

The capsule can be soluble or insoluble in gastric juice. Preferably, the capsule does not dissolve in gastric juice, but dissolves in the environment of the duodenum and upper ileum. The pH of gastric juice is strongly acidic, approximately pH 1-3. The pH of the duodenum is close to neutral, pH 6-6.5, and the pH of the ileum is neutral to slightly basic, pH 7-8. Thus a capsule, which is insoluble in gastric juice and soluble in the duodenum or ileum should be insoluble at low pH (1-3) and soluble at around neutral pH (6-8).

### Combination Products

The compounds of the invention can also be included in a combination product as herein described.

In one embodiment the combination product comprises; (A) a compound or a pharmaceutical composition according to the invention, and (B) a monoacylglycerol-lipase-inhibitor for simultaneous, successive or separate administration.

Monoacylglycerol-lipase inhibitors can be selected from, but are not limited to, the group consisting of JZL184, CAY10499, URB754, OMDM169, URB602, Disulfuram, Tetrahydrolipstatin, N-arachidonyl maleimide, Isopropyl dodecylfluorophosphonate (IDFP), Oxiran-2-ylmethyl (5Z,8Z,11Z,14Z)-icosa-5,8,11,14-tetraenoate, Tetrahydro-2 H-pyran-2-ylmethyl (5Z,8Z,11Z,14Z)-icosa-5,8,11,14-tetraenoate, Cetilistat (ALT-962), and GT-398-255.

In a preferred embodiment the monoacylglycerol-lipase inhibitor is selected from the group consisting of JZL184, CAY10499, URB602, and OMDM169.

In a further embodiment the combination product comprises; (A) a compound or a pharmaceutical composition according to the invention, and (B) a monoacylglycerol-acyltransferase-inhibitor, for simultaneous, successive or separate administration.

In a preferred embodiment the monoacylglycerol-acetyltransferase inhibitor is sphingosine.

In another embodiment the combination product comprises; (A) a compound or a pharmaceutical composition according to the invention, and (B) another therapeutic agent that is useful in the treatment of metabolic syndrome, diabetes type 2, obesity and/or insulin resistance, for simultaneous, successive or separate administration. The therapeutic agent of (B) can be selected from, but are not limited to, the group consisting of sulphonylurea, biguanides, meglitinides, α-glucosidase inhibitors, and DPP-4 inhibitors.

In a further embodiment the combination product comprises; (A) a compound or a pharmaceutical composition according to the invention, and (B) another therapeutic agent that is useful in the treatment of cardiovascular disease.

The invention also provides methods for the prophylaxis and/or treatment of diabetes-2, obesity, insulin resistance, and cardiovascular disease comprising administering an effective amount of a compound according to the invention to a subject in need thereof. Preferably the subject is a human.

Generally, suitable dosage levels of a compound according to the invention are in the order of about 100-5000 mg, such as 500-5000 mg, for example 500-4000 mg, such as 500-3000 mg, for example 500-2000 mg. One or more dosages may be administered per day. A daily dosage can thus be up to 5 g/person, such as up to 10 g/person, for example up to 15 g/person, such as up to 20 g/person, for example 25 g/person for a subject of 70 kg. These values can be converted into dosages per kg/body weight per day.

Most preferred is a dose of 1000 mg of a compound according to the invention. For 2-oleylglycerol a preferred dosage is 2 g/dosage. For other compounds of the invention, a dosage giving the same dosage in moles is preferred. For example for 1,3-dioctoyl-2-oleyoglycerol, an equivalent dosage is 3.5 g/dosage.

Such a dosage could preferably be administered per meal. The dosage can be administered before, after or together with a meal.

### Food and Feed Products

In certain aspects, the invention relates to a food or animal feed product comprising a compound selected from the group consisting of

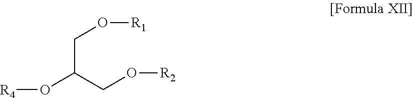

wherein R₁ and R₂ are individually selected from the group consisting of H, and C2-C8 acyl groups,
and wherein R₄ is selected from the group consisting of a C16 or C18 acyl group.

The term food or animal feed product includes dietary supplements and beverages.

Preferably R₁ and R₂ are linear and saturated, as most edible short chain carboxylic acids are linear and saturated. More preferably one of R₁ and R₂ is CH₂-CH₃ and the other is —H. Still more preferably R₁ and R₂ are CH₂-CH₃. Preferred compounds that are 1, and or 3 substituted include 1-acetyl-2-oleoylglycerol and even more preferably 1,3-diacetyl-2-oleoylglycerol.

Compounds that are acylated or acetylated in the 1 and or 3 position are degraded in the stomach to glycerols that are only substituted in the 2-position. This protects the compounds as they pass through the stomach so that more of the active 2-substituted glycerols reach the intestine and activate GPR119. Furthermore, compounds of the invention that are substituted in both the 1- and the 3-positions are oils that can be readily mixed with other oils and are oils that can be used for cooking. Thus it is expected that essentially pure 1,3-acetyl-2-oleylglycerol can be used as a major fraction of salad oil and can be used in an essentially pure form as a cooking or frying oil.

In other embodiments, R₁ and/or R₂ is —H.

Preferably, R₄ is linear. In a preferred embodiment, R₄ is unsaturated. In certain embodiments R₄ is C16 acyl group comprising 0, 1 or 2 double bonds. Examples of such R₄ groups include palmitic and palmitoleic. In other, preferred embodiments, R₄ is C18 acyl group comprising 0, 1, 2, 3, or 4 double bonds. Examples of such R₄ groups include oleic, linoleic, alpha-linoleic, elaidic, gamma linoleic, or stearidonic, preferably oleic. 2-palmitoyl-glycerol has been shown to be an activator of GPR119 by the present inventors.

A compound according to the invention can be incorporated into a food product. The food product can be any food product. Examples of food products are; processed food items, such as bread, diary products, such as yoghurt, smoothies, cheese and ice cream, non diary products, dietary products, spreads, products for diabetics, and salad oil. Also included are high fat products such as mayonnaise, butter, margarine, oils, such as salad oil, cooking oil, and frying oil. Further examples of food products include cakes, cookies, and snacks.

The compound of the invention may amounts to a minimum of 10 weight % of other fats in the product, such as at least 20 weight %, for example at least 30%, such as at least 40%, for example at least 50%, such as at least 60%, for example at least 70%, such as at least 80%, for example at least 90%, such as essentially 100%.

In such high-fat products, the compounds of the invention can serve as low calorie fat substitutes, as the compounds of the invention, in particular glycerols with C18 (such as oleic) in the 2 position and short chain acyl groups in the 1 and 3 positions contain less calories than fatty triglycerides. Such compounds are oils and can be readily mixed with other oils and fats. Such products of course also have an effect on the release of gastric hormones.

A compound according to the invention can be incorporated into an animal feed product, for example a feed product for dogs or cats.

The food and feed products of the invention may reduce the risk or likelihood or extent of dyslepidemia, obesity, type 2 diabetes, metabolic syndrome, and/or may served to release gastric hormones and/or regulate glucose in the blood.

In certain embodiments the food and/or food product may provide one or more of the following effects on an individual eating the food or feed product:

1) Help sustain energy
2) Help control appetite
3) Help control blood sugar levels
4) Reduce the risks associated with metabolic syndrome
5) Reduce the risk associated with obesity and diabetes
6) Reduce the risk associated with diabetes
7) Help to maintain healthy glucose and fat metabolism
8) Help to normalise production and release of GLP1 necessary for healthy glucose and fat metabolism.

The invention thus relates to a method of achieving one or more of the cited effects in an individual in need thereof by providing the individual with an effective amount of the food or feed product of the invention.

In certain embodiments and if the compound incorporated into a food or animal feed product is 1-oleoylglycerol, 1-linoleoylglycerol, 2-oleoylglycerol with short chain esters (R₃=short chain alkyls), or 2-linoleoylglycerol with short chain esters (R₃=short chain alkyls), the food product is preferably a low fat product wherein said compound amount to a minimum of 10 weight % of other fats.

In one embodiment the compound in the food product or animal feed product is encapsulated in a capsule material which is soluble in gastric juice.

In another embodiment the compound in the food product or animal feed product is encapsulated in a capsule material which is insoluble in gastric juice. This ensures that the compounds of the invention are released in the duodenum or ileum.

### EXAMPLES

### Example 1

### In Vitro Experiments with GPR119-Expressing Cells

GPR119 receptor signalling experiments were carried out using COS-7 cells transiently transfected with the human GPR119 receptor. The COS-7 cells were grown at 10% CO2 and 37° C. in Dulbecco's modified Eagle's medium with glutamax (Gibco, Cat. No 21885-025) adjusted with 10% fetal bovine serum (FBS), 180 u/ml penicillin and 45 ug/mL streptomycin (PenStrep). Transfection of the COS-7 cells was performed by the calcium phosphate precipitation method. The cells were seeded in 24 well plates (1.5×10⁵ cells/well) one day after transfection, and were subsequently incubated for 24 hours with 2 μCi/ml of 3H-adenine in 0.5 ml growth medium per well. At the day of the cAMP measurement, the cells were washed twice in HBS buffer (25 mM Hepes, pH 7.2, supplemented with 0.75 mM NaH2PO4, 140 mM NaCl and 0.05% (w/v) bovine serum albumin), and 0.5 ml HBS buffer supplemented with 1 mM of the phosphodiesterase inhibitor IBMX (3-isobutyl-1-methylxanthine, Sigma chemicals Co., St. Louis, Mo., USA) was added together with increasing concentrations of the different compounds (2OG, 1OG, OEA, oleic acid or 2-PG). After 25 min incubation at 37° C., the cells were placed on ice, the medium was removed, and the cells were lysed in 1 ml of 5% (w/v) trichloroacetic acid, supplemented with 0.1 mM cAMP and 0.1 mM ATP for 30 min. The lysate mixtures were loaded onto Dowex columns (Bio-Rad, Hercules, Calif., USA), which were washed with 2 ml of water and placed onto the top of alumina columns (Sigma) and washed again with 10 ml of water. The alumina columns were eluted with 6 ml of 0.1 M imidazole into 15 ml scintillation fluid (Highsafe III). Columns were re-used up to 15 times. Dowex columns were regenerated by adding 10 ml of 2 N HCl followed by 10 ml of water; the alumina columns were regenerated by adding 2 ml of 1 M imidazole, 10 ml of 0.1 M imidazole, and finally 5 ml of water. Determinations were made in duplicate.

GPR119 receptor signalling experiments were carried out using CHO-K1 cells transiently transfected with the human GPR119 receptor. CHO-K1 cells expressing GPR119 grown to mid-log phase prior to the test in culture media without antibiotics and supplemented with doxycycine (final concentration 200 ng/ml) were detached be gentle flushing with PBS-EDTA (5 mM EDTA), recovered by centrifugation and resuspended in Ham's F12 culture medium containing 10% FCS and no antibiotic. Cells were counted, centrifuged in a 50 ml Falcon tube and resuspended in KRH-IBMX (5 mM KCL, 1.25 mM MgSO₄, 124 mM NaCl, 25 mM HEPES, 13.3 mM glucose, 1.25 mM KH₂PO₄, 1.45 mM CaCl₂, 0.6 mg/ml BSA and 10 mg/ml Phenol red, pH 7.4) at a concentration of 6.25×10⁵ cells/ml. Cells were then filled in 96 well plates (total vol 24 μl/well) and stimulated with agonists in serial dilution for 30 min. cAMP was then measured using a HTRF kit from CisBio International (cat no. 62AM2PEB). Determinations were made in duplicate.

### Results:

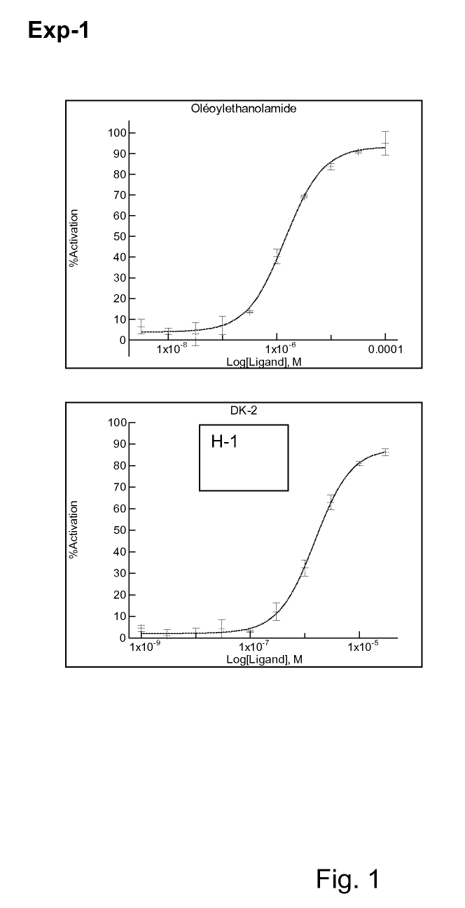

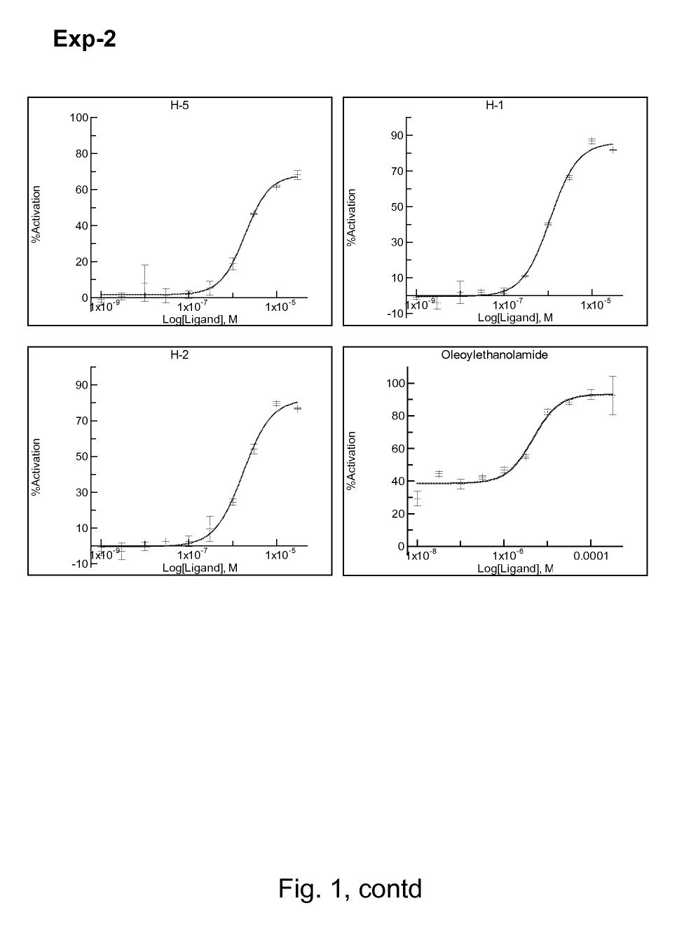

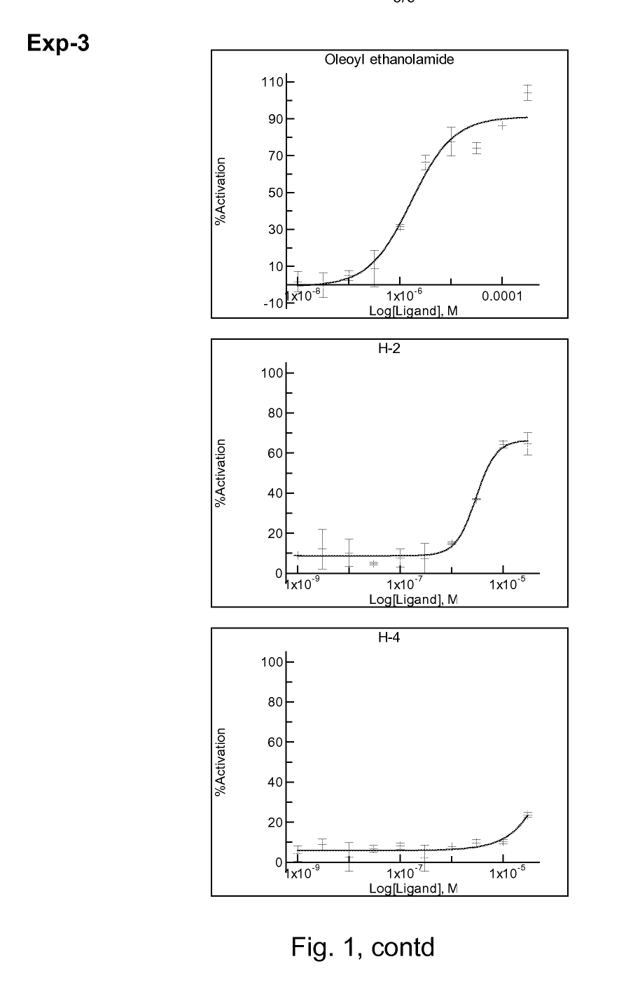

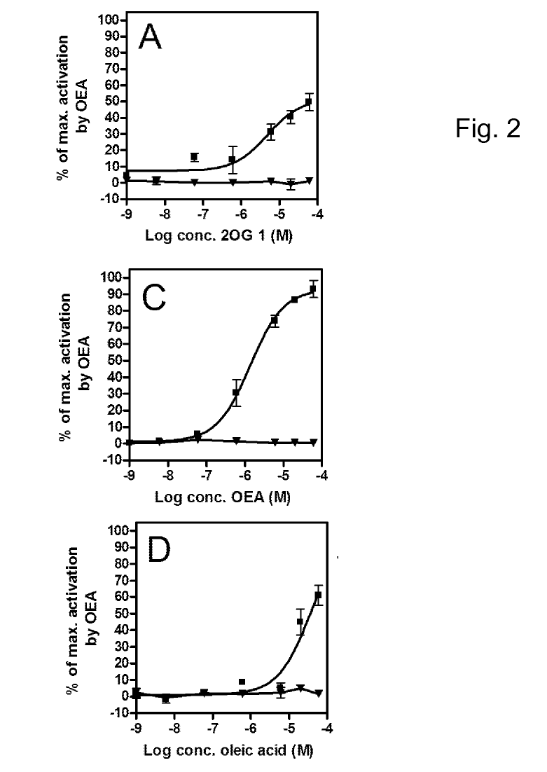

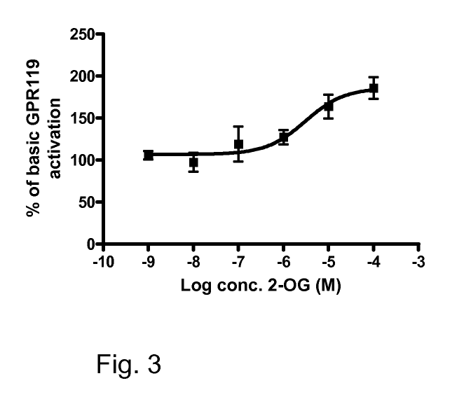

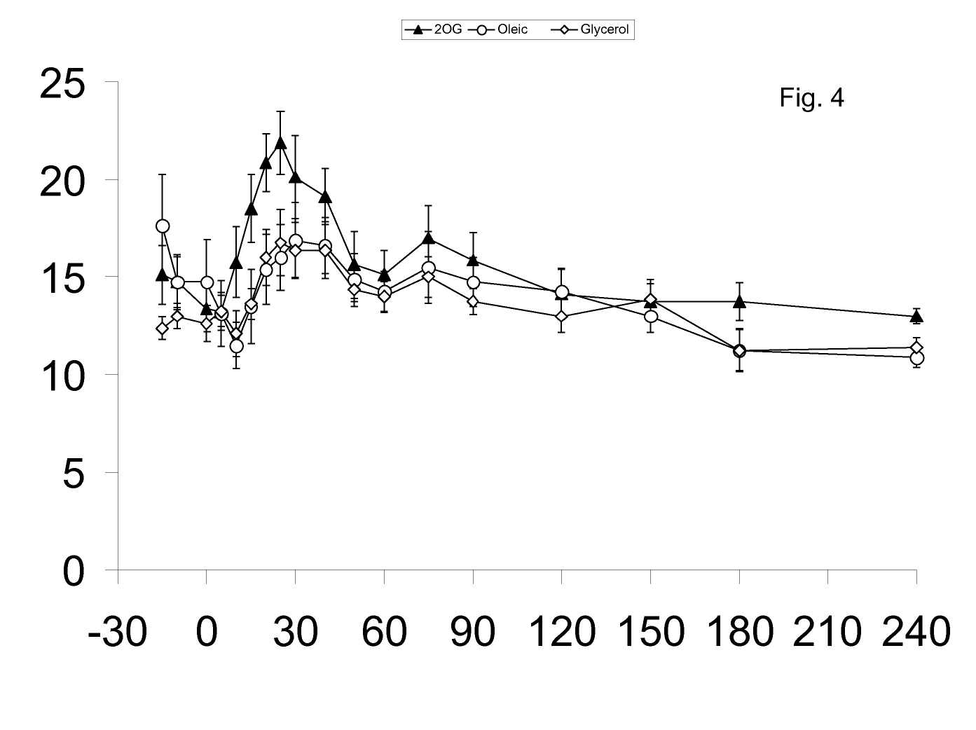

2OG and some of its structural analogues were found to increase cAMP formation in CHO-K1 cells transiently expressing the GPR119 (FIG. 1). 2OG was found to have and EC50 nearly as low as that seen for the recognised GPR119 agonist, oleoylethanolamide (Table 1). 2OG was also tested in another laboratory using COS-7 cells transiently expressing GPR119 (FIG. 2). Also in these cells was 2OG nearly as potent as oleoylethanolamide (FIG. 2). Thus 2OG can readily activate GPR119 and appear to be a full agonist.

TABLE 1
EC-values (μM) for activation of GPR119 in two different expression
systems, CHO-K1-cells and COS-7-cells. In both cell types the cAMP
formation was quantified.

| | | | CHO- | COS-cells |
|---|---|---|---|---|
| | CHO-exp-1 | CHO-exp-2 | exp-3 | (n = 3) |
| Oleoylethanolamide | 1.43 | 4.65 | 1.67 | 1.4 |
| 2-Oleoylglycerol | 1.55 | 1.12 | | 2.8 |
| oleic acid | | | >30 | 32.6 |

Table 2. shows EC50 values (microM) for activation of GPR119 in transiently transfected COS-7-cells (as described previously) by four different compounds using the assay described above. Data for 2-OG are also shown in FIG. 3.

TABLE 2

| Compound | EC50 |
|---|---|
| 2OG | 4.2 (mean, n = 7) |
| oleic acid | >30 (n = 2) |
| 2OG-ether | 7.9 (mean, n = 3) |
| 2-palmitolyglycerol (2PG) | >30 (n = 2) |

### Example 2A

### In Vivo Experiment

Six healthy male volunteers that have fasted for 10 hours, are given enteral feeding by a duodenal tube. Each volunteer is given four different liquid meals (bolus, 55-65 ml) on four different days (A-D)
Day A: 2OG (2 g) in 50 ml glycerol+5 ml ethanol
Day B: oleic acid (1.54 g) in 50 ml glycerol+5 ml ethanol (=control Day A)
Day C: 2OG (2 g)+glucose (10-20 g in 10 ml water) in 50 ml glycerol+5 ml ethanol
Day D: oleic acid (1.54 g)+glucose (10-20 g in 10 ml water) in 50 ml glycerol+5 ml ethanol (=control Day C)
Blood samples are collected at the following time points (min): −15, −10, 0, 10, 20, 30, 40, 50, 60, 75, 90, 120, 150, 180, 240
From duodenal lumen is collected 1 ml sample (at 15 and 30 min)
In serum is measured: insulin and C-peptide
In plasma is measured: glucose, GL P-1, glucose-dependent insulinotrphic polypeptide (GIP), glucagon, peptide-YY, cholecystokinin,
In duodenal sample is measured: bilirubin
The methods for these assays have been described previously [18].

### Example 2B

### Clinical Experiment

8 healthy male volunteers (20-30 years, BMI 20-25) that had fasted 10 hours, were given enteral feeding by duodenal tube. The liquid meals were prepared 5 min before use. Each volunteer was given three different liquid meals (clear solution, bolus 55 ml) on three different days (A-C).

Day A: 2OG (2 g) in 50 ml glycerol+5 ml ethanol
Day B: oleic acid (1.54 g) in 50 ml glycerol+5 ml ethanol
Day C: 50 ml glycerol+5 ml ethanol
Blood Samples were collected at the following points (min): −15, −10, 0, 10, 20, 30, 40, 50, 60, 75, 90, 120, 150, 180, 240.

In serum was measured: insulin and C-peptide

In plasma was measured: glucose, tGLP-1, Cholecystokinin (CCK).

The methods for these assays have been described previously (ref 18).

### Results:

As can bee seen from FIG. 4, during the first 25 minutes there was a significant increased levels of tGLP-1 in the plasma of the individuals as a response to the duodenal bolus of 2 g of 2OG, compared to both vehicle and to oleic acid. The 1.54 g of oleic acid is in mol amount equivalent to the 2 g 2OG, and this amount of oleic acid did not increase plasma levels of GLP-1. As expected, there were no effect of the increased level of tGLP-1 on insulin or c-peptide levels (data not shown), since these persons were fasted when the bolus meal were given. GLP-1 is an incremental hormone that will increase insulin release only when plasma glucose levels are elevated (ref. 3). The plasma levels of glucose started in all three groups to increase after 25 min (no difference between groups, data not shown) due to gluconeogenesis from the glycerol in the vehicle. Likewise no difference was seen in plasma cholecystokinin levels (data not shown).

Clearly, 2OG in the intestine can stimulate GLP-1 release in humans probably via activation of GPR119 in intestinal L-cells.

### REFERENCES

1. Diagnosis and classification of diabetes mellitus. Diabetes Care 31 Suppl 1: S55-S60, 2008.
2. Economic costs of diabetes in the U.S. In 2007. Diabetes Care 31: 596-615, 2008.
3. Hoist J J, Deacon C F, Vilsboll T, Krarup T, and Madsbad S, Glucagon-like peptide-1, glucose homeostasis and diabetes. Trends Mol. Med. 14: 161-168, 2008.
4. Flint A, Raben A, Astrup A, and Hoist J J, Glucagon-like peptide 1 promotes satiety and suppresses energy intake in humans. J. Clin. Invest 101: 515-520, 1998.
5. Zander M, Madsbad S, Madsen J L, and Hoist J J, Effect of 6-week course of glucagon-like peptide 1 on glycaemic control, insulin sensitivity, and beta-cell function in type 2 diabetes: a parallel-group study. Lancet 359: 824-830, 2002.
6. Nauck M, Stockmann F, Ebert R, and Creutzfeldt W, Reduced incretin effect in type 2 (non-insulin-dependent) diabetes. Diabetologia 29: 46-52, 1986.
7. Funnell M M, The therapeutic role of incretin mimetics and DPP-4 inhibitors. Diabetes Educ. 35 Suppl 1: 12S-17S, 2009.
8. Chu Z L, Carroll C, Alfonso J, Gutierrez V, He H, Lucman A, Pedraza M, Mondala H, Gao H, Bagnol D, Chen R, Jones R M, Behan D P, and Leonard J, A role for intestinal endocrine cell-expressed g protein-coupled receptor 119 in glycemic control by enhancing glucagon-like Peptide-1 and glucose-dependent insulinotropic Peptide release. Endocrinology 149: 2038-2047, 2008.
9. Overton H A, Fyfe M C, and Reynet C, GPR119, a novel G protein-coupled receptor target for the treatment of type 2 diabetes and obesity. Br. J. Pharmacol. 153: s76-s81, 2008.
10. Lauffer L M, lakoubov R, and Brubaker P L, GPR119 is essential for oleoylethanolamide-induced glucagon-like peptide-1 secretion from the intestinal enteroendocrine L-cell. Diabetes, 2009.
11. Overton H A, Babbs A J, Doel S M, Fyfe M C, Gardner L S, Griffin G, Jackson H C, Procter M J, Rasamison C M, Tang-Christensen M, Widdowson P S, Williams G M, and Reynet C, Deorphanization of a G protein-coupled receptor for oleoylethanolamide and its use in the discovery of small-molecule hypophagic agents. Cell Metabolism 3: 167-175, 2006.
12. Soga T, Ohishi T, Matsui T, Saito T, Matsumoto M, Takasaki J, Matsumoto S, Kamohara M, Hiyama H, Yoshida S, Momose K, Ueda Y, Matsushime H, Kobori M, and Furuichi K, Lysophosphatidylcholine enhances glucose-dependent insulin secretion via an orphan G-protein-coupled receptor. Biochemical and Biophysical Research Communications 326: 744-751, 2005.
13. Rodriguez de Fonseca F, Navarro M, Gómez R, Escuredo L, Nava F, Fu J,
Murillo-Rodriguez E, Giuffrida A, LoVerme J, Gaetani S, Kathuria S, Gall C, and Piomelli D, An anorexic lipid mediator regulated by feeding. Nature 414: 209-212, 2001.
14. Nielsen M J, Petersen G, Astrup A, and Hansen H S, Food intake is inhibited by oral oleoylethanolamide. J. Lipid Res. 45: 1027-1029, 2004.
15. Hansen H S and Diep T A, N-acylethanolamines, anandamide and food intake. Biochem. Pharmacol. in press.: 2009.
16. Lan H, Vassileva G, Corona A, Liu L, Baker H, Golovko A, Abbondanzo S, Hu W, Yang S, Ning Y, Del Vecchio R, Poulet F, Layerty M, Gustafson E, Hedrick J, and Kowalski T, GPR119 Is Required For Physiological Regulation of Glucagon-Like Peptide-1 Secretion but Not for Metabolic Homeostasis. J. Endocrinol., 2009.
17. Fu J, Oveisi F, Gaetani S, Lin E, and Piomelli D, Oleoylethanolamide, an endogenous PPAR-α agonist, lowers body weight and hyperlipidemia in obese rats. Neuropharmacol. 48: 1147-1153, 2005.
18. Hojberg P V, Vilsboll T, Zander M, Knop F K, Krarup T, Volund A, Hoist J J, and Madsbad S, Four weeks of near-normalization of blood glucose has no effect on postprandial GLP-1 and GIP secretion, but augments pancreatic B-cell responsiveness to a meal in patients with Type 2 diabetes. Diabet. Med. 25: 1268-1275, 2008.
19. Pawongrat R, Xu X, and H-Kittikun A, Synthesis of monoacylglycerol rich in polyunsaturated fatty acids from tuna oil with immobilized lipase AK. Food Chem. 104: 251-258, 2007.
20. Da Silva M A, Medeiros V C, Langone M A, and Freire D M, Synthesis of monocaprin catalyzed by lipase. Appl. Biochem. Biotechnol. 105-108: 757-767, 2003.
21. Ahren B, Islet G protein-coupled receptors as potential targets for treatment of type 2 diabetes. Nat. Rev. Drug Discov. 8: 369-385, 2009.

1.-95. (canceled)

96. A method of treatment of diabetes-2, the method comprising administering to an individual in need thereof a therapeutically effective amount of a compound having the general formula 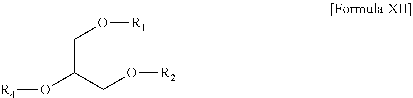
wherein R₁ and R₂ are individually selected from the group consisting of H, C2-C8 acyl groups, and 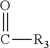
wherein R₃ is selected from alkyl groups comprising 1-12 carbon atoms and wherein R₄ is selected from the group consisting of a C16 or C18 acyl or alkyl group.

97. The method according to claim 96, wherein R₁ and R₂ are linear and saturated.

98. The method according to claim 96, wherein R₃ is an alkyl group consisting of 1-12 carbon atoms.

99. The method according to claim 96, wherein R₄ is linear.

100. The method according to claim 96, wherein R₄ is unsaturated.

101. The method according to claim 96, wherein R₄ is a C16 acyl group comprising 0, 1 or 2 double bonds.

102. The method according to claim 96, wherein R₄ is a C18 acyl group comprising 0, 1, 2, 3, or 4 double bonds.

103. The method according to claim 99, wherein R₄ is oleic, linoleic, alpha-linoleic, elaidic, gamma linoleic, or stearidonic.

104. The method according to claim 96, wherein the compound is 2-oleoyl-glycerol.

105. The method according to claim 96, wherein the compound is 1-oleoyl-glycerol.

106. The method according to claim 96, wherein the compound is 1-acetyl-2-oleoylglycerol.

107. The method according to claim 96, wherein the compound is 1,3-diacetyl-2-oleoylglycerol.

108. The method according to claim 96, wherein the compound is an agonist of G-protein coupled receptor 119.

109. A capsule comprising a compound according to claim 96 or a solvate of said compound.

110. The capsule according to claim 109 wherein the capsule is made of one or several from the group consisting of gelatine, a plant based gelling substance, modified starch, modified cellulose, and derivatives of any of these.

111. The capsule according to claim 109, wherein the capsule is soluble in gastric juice.

112. The capsule according to claim 109, wherein the capsule is insoluble in gastric juice.

113. The capsule according to claim 109 wherein the capsule is soluble in the small intestine.

114. The capsule according to claim 109 wherein the capsule is soluble in the duodenum and upper ileum.

115. The capsule according to claim 109 wherein the capsule is soluble in the jejunum and ileum.

116. The capsule according to claim 109, wherein the capsule is insoluble at low pH (1-3) and soluble around neutral pH (6-8).

117. The capsule according to claim 109 coated for delayed release.

118. The capsule according to claim 109 formulated for sustained and/or controlled release.

119. The capsule according to claim 109 wherein the capsule has an enteric coating.

120. The capsule according to claim 119 wherein the enteric coating is selected from the group consisting of: Cellulose acetate phthalate (CAP), Methyl acrylate-methacrylic acid copolymers, Cellulose acetate succinate, Hydroxy propyl methyl cellulose phthalate, Hydroxy propyl methyl cellulose acetate succinate (hypromellose acetate succinate), Polyvinyl acetate phthalate (PVAP), and Methyl methacrylate-methacrylic acid copolymers.

121. A method of activating G-protein coupled receptor 119 by administering a compound according to claim 96 to a cell expressing said receptor.

122. A method of stimulating the release of GLP-1 in a subject in need thereof comprising administering to said subject a therapeutically effective amount of a compound according to claim 96.

123. The method according to claim 122, wherein the stimulation of the release of GLP-1 occurs in the duodenum or in the ileum.

124. The method according to claim 122, wherein the stimulation of the release of GLP-1 occurs in the pancreas of the subject.

125. The method according to claim 122, wherein the stimulation of the release of GLP-1 occurs in the brain.

126. A food or animal feed product comprising a compound according to claim 96.

127. The food product according to claim 126, in the form of bread, snacks, cookies, energy bars, dairy products, non-dairy products, spread, mayonnaise, butter, margarine, oil, cooking oil, frying oil, and salad oil.

128. The food or animal feed product according to claim 126, wherein said compound amounts to a minimum of 10 weight % of other fats in the product.

129. The food or animal feed product according to claim 126, wherein said compound is encapsulated in a capsule material which is soluble in gastric juice.

130. The food or animal feed product according to claim 126, wherein said compound is encapsulated in a capsule material which is insoluble in gastric juice.
